(19) United States
(12) Patent Application Publication
(10) Pub. No.: US 2017/0252311 A1
(43) Pub. Date: Sep. 7, 2017

(54) SPHINGOSINE KINASE INHIBITORS AND CERAMIDE FOR MAINTENANCE THERAPY OF GLIOBLASTOMA

(71) Applicant: SignPath Pharma Inc., Quakertown, PA (US)

(72) Inventors: Lawrence Helson, Quakertown, PA (US); Laura A. Sordillo, New York, NY (US); Peter Sordillo, New York, NY (US)

(21) Appl. No.: 15/439,405

(22) Filed: Feb. 22, 2017

Related U.S. Application Data
(60) Provisional application No. 62/302,542, filed on Mar. 2, 2016.

(51) Int. Cl. A61K 31/164; A61K 31/137; A61K 31/661; A61K 31/12; A61K 31/4706; A61K 31/4745; A61K 31/05; A61K 31/506; A61K 31/4164; A61K 31/426; A61K 31/343; A61K 31/44; A61K 31/167; A61K 9/127; A61K 31/133

(52) U.S. Cl. CPC A61K 31/164; A61K 31/133; A61K 31/137; A61K 31/661; A61K 31/12; A61K 31/4706; A61K 31/4745; A61K 31/05; A61K 31/506; A61K 31/4164; A61K 31/426; A61K 31/343; A61K 31/44; A61K 31/167; A61K 9/127

(57) ABSTRACT

The present invention includes methods of preventing a ceramide-sensitive cancer comprising: identifying a subject that has been treated for the ceramide-sensitive cancer; and providing to the subject an effective amount of a combination of a sphingosine kinase inhibitor and a ceramide inducing agent or a ceramide analog in an amount sufficient to prevent or reduce the recurrence of the ceramide-sensitive cancer.

### CROSS-REFERENCE TO RELATED APPLICATIONS

This application claims priority to U.S. Provisional Application Ser. No. 62/302,542 filed Mar. 2, 2016, the entire contents of which are incorporated herein by reference.

### TECHNICAL FIELD OF THE INVENTION

The present invention relates in general to the field of maintenance following a treatment for glioblastoma, and more particularly, to compositions and methods for preventing the recurrence of glioblastoma.

### STATEMENT OF FEDERALLY FUNDED RESEARCH

None.

### BACKGROUND OF THE INVENTION

Without limiting the scope of the invention, its background is described in connection with treatments for glioblastoma.

One such method of treatment is taught in U.S. Pat. No. 8,945,563, issued to Auf Der Maur, et al., entitled “Method for treating glioblastoma using antibodies binding to the extracellular domain of the receptor tyrosine kinase ALK.” Briefly, these inventors are said to teach an antibody specific for human ALK (Anaplastic Lymphoma Kinase), in particular a scFv, a nucleic acid sequence encoding it, its production and use as a pharmaceutical, for diagnostic purposes, and the local treatment of glioblastoma.

Another method is taught in U.S. Pat. No. 8,518,698, issued to Sugaya, et al., entitled “Method of promoting apoptosis of glioblastoma tumor cells.” Briefly, these inventors are said to teach a method of promoting apoptosis of human glioblastoma multiforme (GBM) tumor cells. The method is said to comprise: isolating GBM tumor cells from a human brain biopsy specimen, isolating human neural stem cells (HNSCs) from the biopsy specimen, transforming the isolated HNSCs with an operative PEX gene, and exposing GBM tumor cells to the transformed HNSCs to promote apoptosis of the tumor cells mediated by the expressed PEX gene.

Yet another method is taught in U.S. Pat. No. 7,931,922, issued to Newmark, et al., entitled “Methods for treating glioblastoma with herbal compositions”. Briefly, these inventors are said to teach methods for treating glioblastoma, by administration of a composition comprising therapeutically effective amounts of supercritical extracts of rosemary, turmeric, oregano and ginger; and therapeutically effective amounts of hydroalcoholic extracts of holy basil, ginger, turmeric, Scutellaria baicalensis, rosemary, green tea, huzhang, Chinese goldthread, and barberry. It is said that this composition modulates gene expression of genes selected from the group consisting of interleukin-1α, interleukin-1β, heme oxygenase 1, aldo-keto reductase family 1 member C2, colony stimulating factor 3, leukemia inhibitory factor, and heat shock 70 kDa protein.

### SUMMARY OF THE INVENTION

In one embodiment, the present invention includes a method of preventing recurrence of a ceramide-sensitive cancer comprising: identifying a subject that has been treated for the ceramide-sensitive cancer; and providing to the subject an effective amount of a combination of a sphingosine kinase inhibitor and a ceramide inducing agent or a ceramide analog in an amount sufficient to prevent or reduce the recurrence of the ceramide-sensitive cancer. In one aspect, the sphingosine kinase inhibitor is selected from at least one of D,L-threodihydrosphingosine (safingol); N,N,N-trimethylsphingosine; fingolimod (FTY720); fingolimod-phosphate; curcumin; antihistamines; chloroquine; mefloquine; resveratrol; nilotinib; dasatinib; imatinib; 5-naphthalen-2-yl-2H-pyrazole-3-carboxylic acid (2-hydroxy-naphthalen-1-ylmethylene)-hydrazide; 4-[4-(4-chloro-phenyl)-thiazol-2-ylamino]-phenol; 2-(3,4-dihydroxy-benzylidene)-benzofuran-3-one; ((2R,3S,4E)-N-methyl-5-(4′-pentylphenyl)-2-aminopent-4-ene-1,3-diol; or 3-(4-chlorophenyl)-adamantane-1-carboxylic acid (pyridin-4-ylmethyl) amide. In another aspect, the subject has been previously treated with radiation therapy, temozolomide, or both. In another aspect, the ceramide inducing agent is selected from at least one of ceramide, N-(4-hydroxyphenyl)retinamide (4-HPR), L-erythro-ceramide, D-threo-ceramide, L-threo-ceramide; C2-Cer isomers, or C2-dihydroceramide (C2-dhCer) isomers. In another aspect, the sphingosine kinase inhibitor and a ceramide inducing agent are provided prior to the recurrence of the ceramide-sensitive cancer. In another aspect, the sphingosine kinase inhibitor and a ceramide inducing agent are provided concurrently. In another aspect, the sphingosine kinase inhibitor and a ceramide inducing agent are adapted for oral, intravenous, enteral, parenteral, intraperitoneal, intramuscular, transcutaneous or subcutaneous administration. In another aspect, the sphingosine kinase inhibitor and a ceramide inducing agent are adapted for immediate, intermediate or extended release. In another aspect, at least one of the sphingosine kinase inhibitor, the ceramide inducing agent, or both, as suspected of causing QT prolongation and the sphingosine kinase inhibitor or the ceramide inducing agent are provided with an amount of liposomes sufficient to prevent the QT prolongation. In another aspect, the ceramide-sensitive cancer is selected from a cancer selected from a brain, a breast, a lung, a glioblastoma, or a pancreatic cancer. In another aspect, the method further comprises the step of identifying a subject that responded at least partially to a first cancer treatment, obtaining a sample of the cancer to determine if the cancer cells are sensitive to ceramide, and selecting the subject for treatment with the sphingosine kinase inhibitor and a ceramide inducing agent or a ceramide analog to inhibit recurrence of the cancer. In another aspect, the sphingosine kinase inhibitor and the ceramide inducing agent or the ceramide analog is curcumin.

In another embodiment, the present invention includes a method of preventing recurrence of a glioblastoma comprising: treating a subject for glioblastoma; and providing to the subject an effective amount of a combination of a sphingosine kinase inhibitor and a ceramide inducing agent or a ceramide analog in an amount sufficient to prevent, slow, or reduce the recurrence of the glioblastoma. In one aspect, the sphingosine kinase inhibitor is selected from at least one of D,L-threodihydrosphingosine (safingol); N,N,N-trimethyl sphingosine; fingolimod (FTY720); fingolimod-phosphate; curcumin; antihistamines; chloroquine; mefloquine; resveratrol; nilotinib; dasatinib; imatinib; 5-naphthalen-2-yl-2H-pyrazole-3-carboxylic acid (2-hydroxy-naphthalen-1-ylmethylene)-hydrazide; 4-[4-(4-chloro-phenyl)-thiazol-2-ylamino]-phenol; 2-(3,4-dihydroxy-benzylidene)-benzofuran-3-one; ((2R,3S,4E)-N-methyl-5-(4′-pentylphenyl)-2-aminopent-4-ene-1,3-diol; or 3-(4-chlorophenyl)-adamantane-1-carboxylic acid (pyridin-4-ylmethyl) amide. In another aspect, the subject has been previously treated with radiation therapy, temozolomide, or both. In another aspect, the ceramide inducing agent is selected from at lease one of ceramide, N-(4-hydroxyphenyl)retinamide (4-HPR), L-erythro-ceramide, D-threo-ceramide, L-threo-ceramide; C2-Cer isomers, or C2-dihydroceramide (C2-dhCer) isomers. In another aspect, the sphingosine kinase inhibitor and a ceramide inducing agent are provided prior to the recurrence of the glioblastoma. In another aspect, the sphingosine kinase inhibitor and a ceramide inducing agent are provided concurrently. In another aspect, the sphingosine kinase inhibitor and a ceramide inducing agent are adapted for oral, intravenous, enteral, parenteral, intraperitoneal, intramuscular, transcutaneous or subcutaneous administration. In another aspect, the sphingosine kinase inhibitor and a ceramide inducing agent are adapted for immediate, intermediate or extended release. In another aspect, at least one of the sphingosine kinase inhibitor, the ceramide inducing agent, or both, as suspected of causing QT prolongation and the sphingosine kinase inhibitor or the ceramide inducing agent are provided with an amount of liposomes sufficient to prevent the QT prolongation. In another aspect, the method further comprises the step of determining if there has been a recurrence of glioblastoma, and if so, changing the combination of sphingosine kinase inhibitor and a ceramide inducing agent or a ceramide analog.

Yet another embodiment includes a method of identifying a drug for preventing or treating a recurrence of a ceramide-sensitive cancer, the method comprising: a) identifying a first set of patients who have been treated to eliminate the ceramide-sensitive cancer; b) administering a combination of a sphingosine kinase inhibitor and a ceramide inducing agent or a ceramide analog in an amount sufficient to prevent or reduce the recurrence of the ceramide-sensitive cancer to a first subset of the patients, and a placebo to a second subset of the patients; c) repeating step a) after the administration of the candidate drug or the placebo; and d) determining if the candidate drug reduces or delays the recurrence of the ceramide-sensitive cancer that is statistically significant as compared to any reduction occurring in the second subset of patients, wherein a statistically significant reduction indicates that the candidate drug is useful for preventing or treating a recurrence of the ceramide-sensitive cancer. In another aspect, the ceramide-sensitive cancer is selected from a cancer selected from a brain, a breast, a lung, a glioblastoma, or a pancreatic cancer. In another aspect, the method further comprises the step of identifying a subject that responded at least partially to a first cancer treatment, obtaining a sample of the cancer to determine if the cancer cells are sensitive to ceramide, and selecting the subject for treatment with the sphingosine kinase inhibitor and a ceramide inducing agent or a ceramide analog to inhibit recurrence of the cancer. In another aspect, the sphingosine kinase inhibitor and the ceramide inducing agent or the ceramide analog is curcumin.

Yet another embodiment of the present invention includes a composition for preventing recurrence of a ceramide-sensitive cancer comprising an effective amount of a combination of a sphingosine kinase inhibitor and a ceramide inducing agent or a ceramide analog in an amount sufficient to prevent or reduce the recurrence of the ceramide-sensitive cancer. In one aspect, the sphingosine kinase inhibitor is selected from at least one of D,L-threodihydrosphingosine (safingol); N,N,N-trimethylsphingosine; fingolimod (FTY720); fingolimod-phosphate; curcumin; antihistamines; chloroquine; mefloquine; resveratrol; nilotinib; dasatinib; imatinib; 5-naphthalen-2-yl-2H-pyrazole-3-carboxylic acid (2-hydroxy-naphthalen-1-ylmethylene)-hydrazide; 4-[4-(4-chloro-phenyl)-thiazol-2-ylamino]-phenol; 2-(3,4-dihydroxy-benzylidene)-benzofuran-3-one; ((2R,3S,4E)-N-methyl-5-(4′-pentylphenyl)-2-aminopent-4-ene-1,3-diol; or 3-(4-chlorophenyl)-adamantane-1-carboxylic acid (pyridin-4-ylmethyl) amide. In another aspect, the ceramide inducing agent is selected from at lease one of ceramide, N-(4-hydroxyphenyl)retinamide (4-HPR), L-erythro-ceramide, D-threo-ceramide, L-threo-ceramide; C2-Cer isomers, or C2-dihydroceramide (C2-dhCer) isomers.

### BRIEF DESCRIPTION OF THE DRAWINGS

For a more complete understanding of the features and advantages of the present invention, reference is now made to the detailed description of the invention along with the accompanying figures and in which:

FIG. 1 is a diagram that shows the three major pathways for the generation of ceramide.

FIG. 2 is a diagram that shows that Sphingosine-1-phosphate is generated from sphingosine by way of ceramide.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ceramide and sphingosine 1-phosphate (S1P) are sphingolipid metabolites with important signaling functions. Ceramides promote apoptosis, whereas S1P favors proliferation, angiogenesis and cell survival. The balance between these opposing signaling functions is known as the sphingolipid rheostat. A shift in this balance toward S1P is seen in glioblastoma (GBM) and other cancers, and results in tumor cell survival and resistance to chemotherapy. Sphingosine kinase (SK), the enzyme responsible for transforming sphingosine into S1P, plays the critical role in modulating the balance between S1P and ceramides. Chemotherapeutic agents or radiation therapy may induce short-term responses in GBM patients by increasing ceramide levels. However, the present inventors recognized that the enzyme SK may cause the increased ceramide to be metabolized to S1P, restoring the abnormally-high S1P to ceramide balance, and that this may be part of the reason for the near-100% recurrence rate of glioblastoma. The present invention includes a novel maintenance therapy with an SK inhibitor, in patients with GBM who have tumor reduction or stable disease after therapy.

Glioblastoma multiforme (GBM) is an aggressive primary brain neoplasm with a median patient survival of only 14.6 months [1, 2]. Presenting symptoms include nausea, vomiting, blurred vision, headaches, and drowsiness. The tumor is particularly resistant to therapy. Standard initial treatment is maximal tumor resection followed by radiation therapy, with simultaneous administration of temozolomide (TMZ), an oral alkylating agent and imidazotetrazine derivative of dacarbazine [3-9]. Recurrent GBMs are most commonly treated with bevacizumab (Avastin), which suppresses angiogenesis, or lomustine, a lipid-soluble, alkylating nitrosourea, which crosses the blood-brain barrier [10-12]. However, these agents are only effective in a small minority of patients, and then only for a few months. Even with newer chemotherapy drugs and advances in surgical methods, overall patient survival rates continue to be extremely poor, and there is no cure for GBM [13-18]. Alternative approaches, such as using immunotherapy, oncogene therapy or molecular targeting agents are being investigated, but so far none have been shown to have a significant impact on response rate or survival [19-22].

Ceramide and Sphingosine-1-Phosphate. Sphingolipids are components of the eukaryotic membrane. The major sphingolipid, sphingomyelin, is found particularly in the membranes of nerve cells. Sphingomyelins can be hydrolyzed by sphingomyelinases to ceramides and phosphorylcholine [23]. Ceramides are an extremely important group of molecules consisting of sphingosine bases and amide-linked acyl chains, which vary in length from C₁₄ to C₂₆.

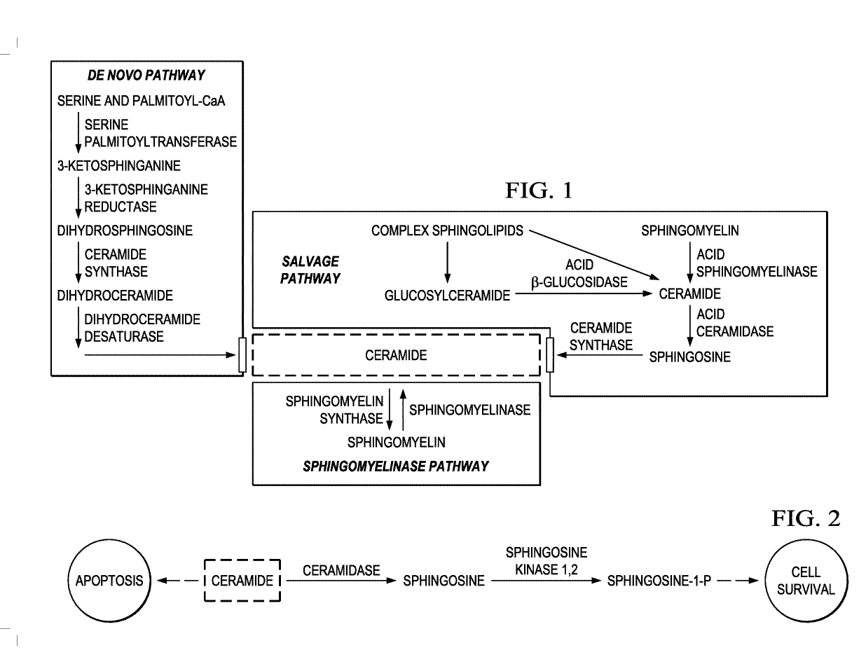

There are three major pathways for the generation of ceramide, the de novo, the sphingomyelinase and the salvage pathway (see FIG. 1) [24, 25]. In the de novo pathway, ceramides are generated from palmitate and serine in a series of steps initiated by the key enzyme serine palmitoyltransferase [24-26]. In the sphingomyelinase pathway, sphingomyelin is hydrolyzed by sphingomyelinase (SMase) [27, 28]. In the salvage pathway, ceramides are formed from the sphingolipid metabolite sphingosine by ceramide synthase [29]. Sphingosine-1-phosphate (S1P) is formed when ceramide is broken down by ceramidase, and the resulting sphingosine molecule is phosphorylated by the enzyme sphingosine kinase [30] (see FIG. 2).

Historically, ceramide and S1P were thought of as merely components of the cell membrane. In the 1990s, however, Obeid et al showed that cell death could be caused by increases in ceramide, and Zhang et al reported on the role of S1P in modulating cellular proliferation [31, 32]. It is now known that ceramide possesses pro-apoptotic signaling functions, whereas S1P plays an important role in proliferation, angiogenesis and cell survival [33-40]. S1P is also responsible for T-cell maturation [41, 42]. The pro-apoptotic functions of ceramides can occur through numerous mechanisms, including increasing protein phosphatase 2A (PP2A), a tumor suppressor, through interaction with microtubule-associated protein 1 light chain 3 beta lipidation (LC3B-II) autophagolysosomes, activation of protein kinase C, and down-modulation of the gene c-myc [43-45]. Ceramide-increasing agents have been shown to cause the destruction of glioblastoma stem cells, the persistence of which are a major cause of glioblastoma recurrence after therapy [46-49].

Sphingosine Kinase. Sphingosine kinase (SK), the signaling enzyme responsible for transforming sphingosine into S1P, plays a critical role in maintaining the balance between ceramides and S1P [50, 51]. In humans, there are two forms of SK, SK1 and SK2. SK1 is mainly located in the cytoplasm and has been studied extensively. It is prominent in white blood cells and in the lung. Cancer cell growth and survival are stimulated by up-regulation of SK1 [52-58]. Increased amounts of SK1 in fibroblasts can cause their malignant transformation to fibrosarcoma [54]. Over-expression of SK1 has been detected in cancers of the breast, prostate, colon, esophagus and lung [59-71]. Benign adenomas of the colon express more SK1 than normal colon cells, and more aggressive metastatic cancers express more SK1 than do cancers that have not metastasized [54]. SK2, on the other hand, is localized to the nucleus, and occurs predominantly in the liver and the kidneys. SK2 appears to have both cell-protective and pro-apoptotic functions [72-74].

Both SK1 and SK2 play important roles in GBM development, progression and resistance to treatment. It has been reported that S1P stimulates invasiveness in human GBM cell lines and tissues through the receptors S1P₁₋₅ [75-79]. On the other hand, Yoshida et al found that while S1P₂ and S1P₃ were increased in patients with GBM, S1P₁ levels were decreased, and lower levels in S1P₁ correlated with poor patient survival [80]. Other studies have shown that SK levels are markedly increased in patients with GBM [81-83]. Van Brocklyn et al showed that patients with GBM and lower SK1 expression survived three times longer than patients with high SK1 [78]. Anelli et al showed that SK1 is over-expressed during hypoxia in U87MG glioma cells [83]. Quint et al investigated the role of SK1, SK2, and of S1P receptors in primary, secondary, and recurrent glioblastoma tissue samples, and showed that SK1 and S1P receptors were overexpressed as much as 44 fold compared to normal brain tissue [84]. With a 25 fold increase, SK2 was highest in primary tumors. Abuhussain et al showed that S1P levels are favored over ceramide levels in patients with glioma, and that increased S1P correlates with increased histologic tumor grade [85]. S1P levels were also nine times higher in areas of tumor compared to areas of normal gray matter, whereas ceramides, in particular C18-ceramide, were five-fold lower.

SK Inhibitors. Two sphingosine kinases inhibitors, D,L-threodihydrosphingosine (safingol) and N,N,N-trimethylsphingosine, have been investigated for a number of years as possible anti-cancer agents. While these agents did not appear to have significant anti-tumor activity alone, there was evidence that they might potentiate the anti-cancer effects of known chemotherapy drugs [86-92]. These inhibitors are not specific to SK, and can affect many protein and lipid kinases [93]. An important SK1 inhibitor, fingolimod (FTY720), has been studied in numerous diseases for more than 20 years, and is now an approved treatment for patients with multiple sclerosis. Fingolimod is phosphorylated by SK2 to fingolimod-phosphate, which binds to S1P receptors and in turn inhibits SK1. Fingolimod is lipophilic and crosses the blood-brain barrier [87, 94-96]. There is evidence for anti-cancer activity of this agent in experimental models [87,97,98]. However, the anti-cancer activity of fingolimod might be muted since it also affects multiple other enzymes which can alter ceramide/S1P balance, including SMase, ceramide synthase, acid ceramidase, S1P lyase and S1P phosphatases [97, 99-100]. For example, fingolimod inhibits both SMase and ceramide synthase, thus partially counteracting the ceramide-increasing effect of SK inhibition [99]. A number of other commonly-used FDA approved agents, including antihistamines, antimalarials, antineoplastics and cardiac medications can affect SK; however, these agents have also been shown to act on multiple other enzymes involved in sphingolipid metabolism.

TABLE 1
Commonly Used Agents with SK Activity.

| | Sphingolipid Enzymes | |
|---|---|---|
| Drug Type | Affected | Reference |
| Fingolimod | ↓SK, ↓ SMase, | 97, 99, 100 |
| | ↓Cer Syn, ↓Acid Cer, | |
| | ↓S1PL, ↓SPP1, | |
| | ↓SPP2 | |
| Tyrosine kinase Inhibitors | ↓SK, ↑Cer Syn | 65, 154-156 |
| (Nilotinib, Dasatinib and | | |
| Imatinib) | | |
| Antimalarials (Chloroquine, | ↓SK, ↑↓Smase, | 101-104 |
| Mefloquine) | ↓SM Syn | |
| | ↓Acid Cer, ↑GCS | |
| Antihistamines | ↓SK, ↑↓SMase | 105, 106 |
| Calcium Channel Blockers | ↓SK, ↓GCS | 105, 107-109 |
| Curcumin | ↓SK, ↑SPT | 110-113 |
| | ↑Cer Syn | |
| Resveratrol | ↓SK, ↑SPT | 114, 115 |
| | ↑Cer Syn | |
| Opioids | ↑SK, ↑SMase | 116, 117 |
| | ↑Cer Syn, ↑SPT | |

Acid Cer: Acid ceramidase; Cer Syn: ceramide synthase; GCS: glucosylceramide synthase; SMase: Sphingomyelinase; SM Syn: Sphingoyelin; SK: sphingose kinase; SPP1, SPP2: S1P phoaphatases; SPT: serine palmitoyltransferase.

More recently, numerous, newer and purer, inhibitors have emerged [90, 118-121]. These agents were found not to affect the broad range of enzymes that the earlier SK inhibitors did. French et al reported anti-tumor activity with three SK1 inhibitors (SKI-I: 5-naphthalen-2-yl-2H-pyrazole-3-carboxylic acid (2-hydroxy-naphthalen-1-ylmethylene)-hydrazide; SKI-II: 4-[4-(4-chloro-phenyl)-thiazol-2-ylamino]-phenol; SKI-V: 2-(3,4-dihydroxy-benzylidene)-benzofuran-3-one)) in a JC mouse mammary adenocarcinoma cell line and in a syngeneic BALB/c mouse solid tumor model of JC mammary adenocarcinoma cells [86]. SKI-II was found to be especially effective in this model. Paugh et al studied a SK1 specific inhibitor ((2R,3S,4E)-N-methyl-5-(4′-pentylphenyl)-2-aminopent-4-ene-1,3-diol (SK1-I), in human leukemic cell lines and acute myelogenous leukemia xenografts, and showed that SK1-I blocked tumor growth and induced apoptosis. Unlike early SK inhibitors, SK1-I does not inhibit SK2, protein kinase B, protein kinase C or other serine/threonine kinases [122]. The selective SK2 inhibitor, ABC294640 (yeliva), has been studied in prostate cancer cell lines and TRAMP-C2 xenografts, and found to cause reduced cell viability and decreased expression of c-myc. This agent is currently part of a phase I clinical trial in advanced solid tumors [123]. Neubauer et al showed that selective targeting of SK2, instead of SK1, could provide additional therapeutic benefits [124]. Schrecengost et al used ABC294640 in xenograft prostate cancer models, and reported that it significantly blocked cancer growth [125].

A few studies of SK inhibitors have been done in GBM. Van Brocklyn et al have shown that SK isoforms play a critical role in the growth and aggressiveness of glioblastoma cells in vitro [78]. They reported that the SK1 inhibitor (2-(p-Hydroxyanilino)-4-(p-chlorophenyl)thiazole) significantly decreased the rate of proliferation in the glioblastoma cell lines U-87 MG, U-1242 MG and M059K [78, 89]. Similarly, Bektas et al used the SK1 inhibitor 2-(p-hydroxyanilino)-4-(p-chlorophenyl)thiazole to promote cancer cell death in the TMZ resistant GBM cell lines U251 and D54MG [89, 126]. Kapitonov et al used the inhibitor SK1-I against LN229 and U373 glioblastoma cell lines, non-established human GBM6 cells, and GBM xenografts, and showed that targeting SK1 inhibits protein kinase B (Akt) signaling, prompts apoptosis, and suppresses tumor development in human GBM cell lines and GBM xenografts [127]. Inhibition of S1P resulted in blocked angiogenesis. Other investigators have reported similar results [128-131]. (Table 2).

TABLE 2
SK inhibitors as single agents in GBM

| Sphingosine Kinase | | | |
|---|---|---|---|
| Inhibitor | Model | Comments | Ref. |
| 2-(p-Hdroxyanilino)-4-(p- | U87MG, U1242MG, | Decreased cellular proliferation | 78 |
| chlorophenyl)thiazole | M059K cell lines | in all three lines. | |
| 2-(p-Hdroxyanilino)-4-(p- | U251MG, D54MG cell | Active in cell lines mde resistant | 126 |
| chlorophenyl)thiazole | lines | to TMZ | |
| N,N-dimethylshingosine | C6 glioma cell line | Suppressed tumor necrosis | 128 |
| (DMS) | | fractor-α induced GTP | |
| | | cyclohydrolase (GTPCH) | |
| | | activity | |
| FTY720 (Fingolimod) | T98G, A172, U87MG, | Induced apoptosis. Activated | 98 |
| | U373MG glioma cell | caspse-6. Caused tyrosine | |
| | lines | dephosphorylation of focal | |
| | | adhesion kinase (FAK) | |
| FTY720 (Fingolimod) | Brain tumor stem cells | Caused BTSC apoptosis. | 130 |
| | (BTSCs) from human | Inactivation of extracellular | |
| | GBM tissue (cell lines | signal-regulated kinases. | |
| | BTSC9, STSC44 and | | |
| | BTSC57) | | |
| FTY720 (Fingolimod) | BTSC xenografts | Reduced tumor size. Increased | 130 |
| | | mouse survival. Augmented | |
| | | efficacy of TMZ. | |
| FTY720 (Fingolimod) | U251MG, SHG44, A172 | Caused apoptosis through FAK | 131 |
| | and U87MG cell | pathway. Reduced of cell | |
| | lines | viability. | |
| FTY720 (Fingolimod) | U251MG xenografts | Inhibited tumor growth. Induced | 131 |
| | | autophagy, apoptosis and | |
| | | necroptosis in vivo. | |
| FTY720 (Fingolimod) | U87MG and U251MG | Deceased invasiveness. Down- | 132 |
| | cell lines | regulation of matrix | |
| | | metalloproteinase-2(MMP-2) | |
| | | and MMP-9 | |
| FTY720 (Fingolimod) | A172, G28 and | Much greater anti-proliferation | 133 |
| | U87MG cell lines | effect than TMZ. | |
| SK1-I ((2R,3S,4E)-- | LN229, U373 cell | Inhibited cell growth and | 127 |
| methyl-5-(4′- | lines | migration. No effect on | |
| pentylphenyl)-2- | | extracellular signal-regulation | |
| aminopent-4-ene-1,3-diol | | kinases. | |
| (BML-258)) | | | |
| SK1-I ((2R,3S,4E)-- | GBM6 cell lines | Reduced cell growth. Reduced | 127 |
| methyl-5-(4′- | | epidermal growth factor- | |
| pentylphenyl)-2- | | stimulated phosphorylation of | |
| aminopent-4-ene-1,3-diol | | Akt. | |
| (BML-258)) | | | |
| SK1-I ((2R,3S,4E)-- | LN229 intracranial | Reduced tumor growth rate. | 127 |
| methyl-5-(4′- | xenografts | Caused apoptosis. Reduced | |
| pentylphenyl)-2- | | angiogenesis. | |
| aminopent-4-ene-1,3-diol | | | |
| (BML-258)) | | | |
| SKI-Ia (N-terminalvariant | U87MG cell line | Blocked angiogenesis. No effect | 85 |
| of SK1) | | on cell survival. | |
| SKI-II | U98G cell line | Decreased proliferation. Caused | 92 |
| | | accumulation of cells at G1. | |
| | | Decreased invasiveness. Blocked | |
| SKI-II | U118MG cell line | expression of urokinase | 134 |
| | | plasminogen activator. | |

Preventing loss of ceramide-induced tumor response. As noted, though ceramides and S1P have opposing signaling functions, they are closely connected. S1P may be dephosphorylated to form sphingosine, and sphingosine then re-acylated to form ceramide. Similarly, sphingosine can be phosphorylated by SK to produce S1P. Cuvillier et al were the first to use the term “sphingolipid rheostat” to describe the balance between ceramide and S1P, and concluded that a shift in this balance plays a role in the determination of the cell's fate [135]. An increase in ceramides predisposes to cell death, whereas excesses of S1P are protective. A number of diseases or disease conditions are associated with abnormalities of the rheostat [136-140]. SK determines whether S1P or ceramide will dominate, and the fate of the cell is determined by the greater relative content of these opposing signaling molecules. Cancer is associated with an increase in S1P within the cell, and with decreases in ceramide [141-145].

SK inhibitors alone will increase ceramide levels, but not as much as when given in combination with an agent which stimulates SMase or ceramide synthase. SK inhibitors have been used in combination with cytotoxic chemotherapy with the goal of increasing ceramides [146-149], and we have previously suggested that increased apoptosis of GBM cells may be achieved using combinations of agents which each increase ceramides [150]. Noack et al used SKI-II with TMZ against the human GBM cell line NCH82, and found that the combination enhanced caspase-3 dependent cell death and autophagy [151]. Similarly, Riccitelli et al showed that an SK1 inhibitor increased chemo-sensitivity to TMZ in a human glioblastoma cell line [152]. Estrada-Bernal et al used FTY720 in combination with TMZ in xenografts of GBM stem cells, and found that tumor volume significantly decreased and mouse survival times increased [130]. Treatment with FTY720 with TMZ resulted in longer survival times compared to FTY720 or TMZ alone. However, the present inventors recognized that the optimal use of SK inhibitors is not as ceramide-inducing agents, but rather in preventing the increased ceramides that are produced after chemotherapy or radiation therapy from being later metabolized to S1P. For example, if this theory is correct, maintenance therapy with SK inhibitors could extend the survival of patients with GBM who have first been treated with radiation therapy and temozolomide and have achieved a response.

The present invention prevents tumor progression, which occurs after response to therapy. Over time, the ceramides that are induced by chemotherapy or radiation therapy are converted to S1P through the actions of SK. Then the excess of S1P over ceramide in the tumor, which was in effect before treatment, is restored, resulting in loss of response, as is typically seen after a short period of time in GBM and other solid tumors. Loss of response to ceramide-inducing agents has been seen in patients who tumors express high levels of SK. For example, patients with estrogen receptor-positive breast carcinoma treated with tamoxifen, an agent which decreases acid ceramidase, had shorter recurrence times if their cancers had higher SK levels [87, 138]. Likewise, patients with head and neck carcinoma with high levels of SK had a much shorter time to progression after radiation therapy [153]. An SK inhibitor might prevent the ceramide from being later converted to S1P, reducing the chance of loss of response. Indeed, one of the reasons that patients with chronic myelogenous leukemia (CML) treated with tyrosine kinase inhibitors (TKIs) have a much lower recurrence rate than do patients with solid tumors or CML patients treated with chemotherapy or interferon, may be because TKIs not only increase ceramide by stimulating ceramide synthase, but also inhibit SK [65, 154-156]. In view of the near-universal tendency of GBM to recur, long-term maintenance therapy with an SK inhibitor may be needed to prevent relapse and progression of disease.

Glioblastoma Pre-Clinical Study.

Glioblastoma Multiforme (GBM), the most common adult primary brain tumor, has poor prognosis with <3% survival after 5 years of diagnosis. Currently, treatment combines chemotherapy, temozolomide (TMZ), radiotherapy and resectional surgery.TMZ is an alkylating agent that induces apoptosis through DNA strand breaks and is considered as the first-line chemotherapeutic agent for GBM. Despite its use, GBM patients commonly exhibit resistance to TMZ treatment, and recurrence following treatment. Chemoresistance include mismatch repair of genes, cell cycle alterations, expression of ATP-dependent drug efflux pumps, epidermal growth factor receptor, intercellular communication through gap junction with activation of EGFR1 that activates AP-1 to increase Cx43 transcription which expression is regulated at the level of transcription in the chemoresistant GBM cells, and decrease of tumor intracellular ceramide with associated increased sphingosine 1phosphate.

The study is conducted in xenotransplanted human brain tumors in nude mice to replicate the clinical situation as closely as possible in order to determine whether Liposomal curcumin and lomustine as second-line therapy followed by maintenance therapy with liposomal curcumin/gilenya (fingolomod) would extend tumor regression compared to treatment with liposomal curcumin/lomustine and no maintenance therapy.

Animals—nude mice maintained under standard conditions of light/darkness, food, age, gender (males). Drugs-temozolamide, lomustine, liposomal curcumin, gilenya. Tumor Cell lines-chemoresistance can be established with 200 μM TMZ for 72 h which increases Cx43 expression in U87 or T98G cells.

1. Treat tumor cells with temozolamide 200 uM for 72 hours.

2. Inoculate subcutaneously 2×10⁶ treated cells into nude mice:

Group (a) untreated control 8 mice;

Group (b) TMZ treated cells 8 mice;

Group (c) TMZ treated cells 8 mice;

Group (d) TMX treated cells 8 mice;

3. When tumors are measurable e.g. >2×2 mm size, the treatment begins, as follows:

Group (b)—treat with liposomal curcumin 20 mg/kg TIW IP for 3 weeks and Lomustin 8 mg/kg per os days #1 and 21 induction, no further therapy.

Group (c)—same as Group (b) induction except continue with 6 weeks of liposomal curcumin @ 20/kg TIW.

Group (d)—same as Group (b) induction, except continue with 6 weeks of Gilenya 2 mg/kg/day per os.

4. Measure survival of each Group (a)-(d), where control untreated group a tumor exceed veterinarians imposed limits (in the US 0 mm diameter), then sacrifice animal. Measure at necropsy, tumors size, histology and weight of mice.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of preventing recurrence of a ceramide-sensitive cancer comprising:
identifying a subject that has been treated for the ceramide-sensitive cancer; and
providing to the subject an effective amount of a combination of a sphingosine kinase inhibitor and a ceramide inducing agent or a ceramide analog in an amount sufficient to prevent or reduce the recurrence of the ceramide-sensitive cancer.

2. The method of claim 1, wherein the sphingosine kinase inhibitor is selected from at least one of D,L-threodihydrosphingosine (safingol); N,N,N-trimethylsphingosine; fingolimod (FTY720); fingolimod-phosphate; curcumin; antihistamines; chloroquine; mefloquine; resveratrol; nilotinib; dasatinib; imatinib; 5-naphthalen-2-yl-2H-pyrazole-3-carboxylic acid (2-hydroxy-naphthalen-1-ylmethylene)-hydrazide; 4-[4-(4-chloro-phenyl)-thiazol-2-ylamino]-phenol; 2-(3,4-dihydroxy-benzylidene)-benzofuran-3-one; ((2R,3S,4E)-N-methyl-5-(4′-pentylphenyl)-2-aminopent-4-ene-1,3-diol; or 3-(4-chlorophenyl)-adamantane-1-carboxylic acid (pyridin-4-ylmethyl) amide.

3. The method of claim 1, wherein the subject has been previously treated with radiation therapy, temozolomide, or both.

4. The method of claim 1, wherein the ceramide inducing agent is selected from at least one of ceramide, N-(4-hydroxyphenyl)retinamide (4-HPR), L-erythro-ceramide, D-threo-ceramide, L-threo-ceramide; C2-Cer isomers, or C2-dihydroceramide (C2-dhCer) isomers.

5. The method of claim 1, wherein the sphingosine kinase inhibitor and a ceramide inducing agent are provided prior to the recurrence of the ceramide-sensitive cancer.

6. The method of claim 1, wherein the sphingosine kinase inhibitor and a ceramide inducing agent are provided concurrently.

7. The method of claim 1, wherein the sphingosine kinase inhibitor and a ceramide inducing agent are adapted for oral, intravenous, enteral, parenteral, intraperitoneal, intramuscular, transcutaneous or subcutaneous administration.

8. The method of claim 1, wherein the sphingosine kinase inhibitor and a ceramide inducing agent are adapted for immediate, intermediate or extended release.

9. The method of claim 1, wherein at least one of the sphingosine kinase inhibitor, the ceramide inducing agent, or both, as suspected of causing QT prolongation and the sphingosine kinase inhibitor or the ceramide inducing agent are provided with an amount of liposomes sufficient to prevent the QT prolongation.

10. The method of claim 1, wherein the ceramide-sensitive cancer is selected from a cancer selected from a brain, a breast, a lung, a glioblastoma, or a pancreatic cancer.

11. The method of claim 1, further comprising the step of identifying a subject that responded at least partially to a first cancer treatment, obtaining a sample of the cancer to determine if the cancer cells are sensitive to ceramide, and selecting the subject for treatment with the sphingosine kinase inhibitor and a ceramide inducing agent or a ceramide analog to inhibit recurrence of the cancer.

12. The method of claim 1, wherein the sphingosine kinase inhibitor and the ceramide inducing agent or the ceramide analog is curcumin.

13. A method of preventing recurrence of a glioblastoma comprising:
treating a subject for glioblastoma; and
providing to the subject an effective amount of a combination of a sphingosine kinase inhibitor and a ceramide inducing agent or a ceramide analog in an amount sufficient to prevent, slow, or reduce the recurrence of the glioblastoma.

14. The method of claim 13, wherein the sphingosine kinase inhibitor is selected from at least one of D,L-threodihydrosphingosine (safingol); N,N,N-trimethylsphingosine; fingolimod (FTY720); fingolimod-phosphate; curcumin; antihistamines; chloroquine; mefloquine; resveratrol; nilotinib; dasatinib; imatinib; 5-naphthalen-2-yl-2H-pyrazole-3-carboxylic acid (2-hydroxy-naphthalen-1-ylmethylene)-hydrazide; 4-[4-(4-chloro-phenyl)-thiazol-2-ylamino]-phenol; 2-(3,4-dihydroxy-benzylidene)-benzofuran-3-one; ((2R,3S,4E)-N-methyl-5-(4′-pentylphenyl)-2-aminopent-4-ene-1,3-diol; or 3-(4-chlorophenyl)-adamantane-1-carboxylic acid (pyridin-4-ylmethyl) amide.

15. The method of claim 13, wherein the subject has been previously treated with radiation therapy, temozolomide, or both.

16. The method of claim 13, wherein the ceramide inducing agent is selected from at lease one of ceramide, N-(4-hydroxyphenyl)retinamide (4-HPR), L-erythro-ceramide, D-threo-ceramide, L-threo-ceramide; C2-Cer isomers, or C2-dihydroceramide (C2-dhCer) isomers.

17. The method of claim 13, wherein the sphingosine kinase inhibitor and a ceramide inducing agent are provided prior to the recurrence of the glioblastoma.

18. The method of claim 13, wherein the sphingosine kinase inhibitor and a ceramide inducing agent are provided concurrently.

19. The method of claim 13, wherein the sphingosine kinase inhibitor and a ceramide inducing agent are adapted for oral, intravenous, enteral, parenteral, intraperitoneal, intramuscular, transcutaneous or subcutaneous administration.

20. The method of claim 13, wherein the sphingosine kinase inhibitor and a ceramide inducing agent are adapted for immediate, intermediate or extended release.

21. The method of claim 13, wherein at least one of the sphingosine kinase inhibitor, the ceramide inducing agent, or both, as suspected of causing QT prolongation and the sphingosine kinase inhibitor or the ceramide inducing agent are provided with an amount of liposomes sufficient to prevent the QT prolongation.

22. The method of claim 13, further comprising the step of determining if there has been a recurrence of glioblastoma, and if so, changing the combination of sphingosine kinase inhibitor and a ceramide inducing agent or a ceramide analog.

23. A method of identifying a drug for preventing or treating a recurrence of a ceramide-sensitive cancer, the method comprising:
a) identifying a first set of patients who have been treated to eliminate the ceramide-sensitive cancer;
b) administering a combination of a sphingosine kinase inhibitor and a ceramide inducing agent or a ceramide analog in an amount sufficient to prevent or reduce the recurrence of the ceramide-sensitive cancer to a first subset of the patients, and a placebo to a second subset of the patients;
c) repeating step a) after the administration of the candidate drug or the placebo; and
d) determining if the candidate drug reduces or delays the recurrence of the ceramide-sensitive cancer that is statistically significant as compared to any reduction occurring in the second subset of patients, wherein a statistically significant reduction indicates that the candidate drug is useful for preventing or treating a recurrence of the ceramide-sensitive cancer.

24. A composition for preventing recurrence of a ceramide-sensitive cancer comprising an effective amount of a combination of a sphingosine kinase inhibitor and a ceramide inducing agent or a ceramide analog in an amount sufficient to prevent or reduce the recurrence of the ceramide-sensitive cancer.

25. The composition of claim 24, wherein the sphingosine kinase inhibitor is selected from at least one of D,L-threodihydrosphingosine (safingol); N,N,N-trimethylsphingosine; fingolimod (FTY720); fingolimod-phosphate; curcumin; antihistamines; chloroquine; mefloquine; resveratrol; nilotinib; dasatinib; imatinib; 5-naphthalen-2-yl-2H-pyrazole-3-carboxylic acid (2-hydroxy-naphthalen-1-ylmethylene)-hydrazide; 4-[4-(4-chloro-phenyl)-thiazol-2-ylamino]-phenol; 2-(3,4-dihydroxy-benzylidene)-benzofuran-3-one; ((2R,3S,4E)-N-methyl-5-(4′-pentylphenyl)-2-aminopent-4-ene-1,3-diol; or 3-(4-chlorophenyl)-adamantane-1-carboxylic acid (pyridin-4-ylmethyl) amide.

26. The composition of claim 24, wherein the ceramide inducing agent is selected from at lease one of ceramide, N-(4-hydroxyphenyl)retinamide (4-HPR), L-erythro-ceramide, D-threo-ceramide, L-threo-ceramide; C2-Cer isomers, or C2-dihydroceramide (C2-dhCer) isomers.
